(19) United States
(12) Patent Application Publication
(10) Pub. No.: US 2009/0136550 A1
(43) Pub. Date: May 28, 2009

(54) MODIFIED RELEASE FORMULATIONS OF DILTIAZEM

(75) Inventors: Subraman Rao Cherukuri, Vienna, VA (US); Revanth Babu Mutyala, Frederick, MD (US); Venkat Ravella, Hyderabad (IN)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Capricorn Pharma Inc., Frederick, MD (US)

(21) Appl. No.: 12/272,421

(22) Filed: Nov. 17, 2008

Related U.S. Application Data
(60) Provisional application No. 61/003,335, filed on Nov. 16, 2007.

(51) Int. Cl. A61K 9/48; A61K 31/554; A61K 9/14

(52) U.S. Cl. 424/401; 514/211.07; 424/497; 424/463

(57) ABSTRACT

Modified release diltiazem compositions and associated methods of preparation and administration are provided.

### CROSS-REFERENCE TO RELATED APPLICATIONS

This application claims the benefit of U.S. Provisional Patent Application No. 61/003,335 filed Nov. 16, 2007, which is incorporated herein by reference in its entirety.

### FIELD OF THE INVENTION

The present invention relates to the field of pharmaceutical sciences, and more specifically to modified release diltiazem formulations.

### BACKGROUND OF THE INVENTION

Diltiazem has been used for many years as an active agent in an immediate release dosage form to control cardiovascular disorders, but it has proven difficult to formulate diltiazem to achieve a modified release profile. This obstacle stems in part because diltiazem hydrochloride is highly water soluble, which makes it difficult to control its release.

Modified release diltiazem formulations are desirable because they can achieve better control of hypertension for a longer period of time compared to immediate release formulations, which often require multiple doses in a single day. Therefore, modified release formulation are more convenient and improve patient compliance. Only a few modified release diltiazem products are available (e.g., Cardizem LA® and Tiazac®).

Cardizem LA® is an extended release tablet containing diltiazem hydrochloride. Tiazac® is an extended release capsule containing diltiazem hydrochloride. The Orange Book lists four patents for Cardizem LA®: U.S. Pat. Nos. 5,288,505; 5,529,791; 6,923,984; and 7,108,866. U.S. Pat. No. 5,529,791 is also listed as the sole patent for Tiazac®.

U.S. Pat. Nos. 5,288,505 and 5,529,791 disclose an extended release diltiazem composition comprising coated beads. The beads include diltiazem and a wetting agent. Col. 2, lines 53-58. The beads are coated with “a microporous membrane comprising a water-soluble or water-dispersible polymer or copolymer, and a pharmaceutically acceptable adjuvant.” Col. 2, lines 18-21. The water-soluble or water-dispersible polymer or copolymer can be a polyacrylate or polymethacrylate of the Eudragit type, such as E30D, ethylcellulose, hydroxypropyl cellulose, and hydroxypropyl methylcellulose. The adjuvant can be a plastifying agent, pigment, filler, wetting agent, lubricant, or antifoaming agent. Col. 3, lines 47-64.

U.S. Pat. No. 7,108,866 discloses that “[i]f the Diltiazem and/or pharmaceutically acceptable salt is not mixed with the wetting agent then the microporous membrane should comprise with suitable adjuvants, a water-dispersible or water-soluble polymer (such as HPMC) and a water-, acid- and base-insoluble polymer of a neutral acrylic polymer such as Eudragit NE30D (a neutral copolymer of acrylic acid ethyl ester and acrylic acid methyl ester) which hydrates the microgranule (including core).” Col. 6, lines 35-42.

U.S. Pat. No. 6,923,984 discloses biologically inactive cushioning beads comprising microcrystalline hydrocarbon wax or a natural wax useful for making compressed sustained release pharmaceutical compositions. Col. 7, lines 39-47.

The present inventors have surprisingly discovered new methodologies for preparing extended release diltiazem. The inventors have also discovered new formulations, which achieve a desirable modified release profile and are also economical to manufacture.

### BRIEF SUMMARY OF THE INVENTION

In one embodiment, a modified release diltiazem pharmaceutical composition is provided comprising:
a) a plurality of individual granules comprising a therapeutically effective amount of diltiazem hydrochloride and one or more pharmaceutically acceptable binders, wherein the individual granules are substantially free of a wetting agent; and
b) a release-modifying coating comprising either 1) one or more neutral acrylate polymers or 2) one or more water-insoluble cellulosic polymers, but not both; and wherein
   i) the individual granules are coated with the release-modifying coating to provide coated granules, or
   ii) the plurality of individual granules are compressed together to provide a core, and the core is coated with the release-modifying coating.

In one embodiment, the therapeutically effective amount of diltiazem hydrochloride is about 60 to about 500 mg.

In one embodiment, the one or more pharmaceutically acceptable binders constitute about 1% to about 15% by weight of the granules. In another embodiment, the one or more pharmaceutically acceptable binders is selected from the group consisting of: acrylate polymers, cellulosic polymers, starch, povidone, sodium starch glycolate, and crospovidone. In another embodiment, the cellulosic polymer is selected from the group consisting of: microcrystalline cellulose, ethylcellulose, propylcellulose, isopropylcellulose, methylcellulose, carboxymethylcellulose, hydroxypropyl cellulose, hydroxypropyl methylcellulose, and hydroxyethyl cellulose.

In another embodiment, the release-modifying coating constitutes about 1% to about 30% or about 5% to about 15% by weight of the pharmaceutical composition.

In one embodiment, the release-modifying coating comprises one or more neutral acrylate polymers selected from the group consisting of: polymers of ethyl acrylate, polymers of methyl methacrylates, polymers of methacrylates, copolymers of ethyl acrylate and methyl methacrylate, and methacrylate copolymers with trimethylammonio-ethylmethacrylate. In another embodiment, the release-modifying coating comprises a copolymer of ethyl acrylate and methyl methacrylate in a ratio of 2:1.

In another embodiment, the release-modifying coating comprises one or more water-insoluble cellulosic polymers selected from the group consisting of: ethylcellulose, methylcellulose, propylcellulose, hydroxyethyl cellulose, and hydroxypropyl methylcellulose. In another embodiment, the release-modifying coating comprises ethylcellulose.

In one embodiment, the coated granules are compressed to form a core. The core, formed from coated or uncoated granules, can be coated with the release-modifying coating and/or a cosmetic coating. In another embodiment, the coated granules are filled into a soft or hard capsule shell.

In one embodiment, the granules are spheroids.

In another embodiment, a process for preparing a modified release diltiazem pharmaceutical composition is provided comprising the steps of:
a) combining a therapeutically effective amount of diltiazem hydrochloride and one or more pharmaceutically acceptable binders, wherein the diltiazem mixture is substantially free of a wetting agent;
b) granulating the diltiazem mixture to provide diltiazem granules;
c) preparing a release-modifying coating comprising either 1) one or more neutral acrylate polymers or 2) one or more water-insoluble cellulosic polymers, but not both; and
   i) coating the plurality of individual granules with the release-modifying coating to form coated granules; or
   ii) compressing the plurality of individual granules together to provide a core, and then coating the core with the release-modifying coating.
      The binders and release-modifying polymers can be selected as described above with reference to the pharmaceutical compositions.

In one embodiment, granulating comprises wet granulation.

In one embodiment, the method further comprises extrusion, spheronization, and/or compression.

In another embodiment, the method further comprises filling a soft or hard capsule shell with the coated granules.

In another embodiment, the method further comprises applying a cosmetic coating.

In another embodiment, we provide a modified release diltiazem pharmaceutical composition prepared by the methods described above.

Additional features, advantages, and embodiments of the invention may be set forth or apparent from consideration of the following detailed description and claims. Moreover, it is to be understood that both the foregoing summary of the invention and the following detailed description are exemplary and intended to provide further explanation without limiting the scope of the invention as claimed.

### BRIEF DESCRIPTION OF THE FIGURES

FIG. 1 compares the in-vitro dissolution of the diltiazem HCl extended release 420 mg capsules described in Example 12A vs. the commercially available diltiazem product Tiazac® extended release 420 mg capsules in pH 1.2 simulated gastric fluid without pepsin.

FIG. 2 compares the in-vitro dissolution of the diltiazem HCl extended release 420 mg capsules described in Example 12A vs. the commercially available diltiazem product Tiazac® extended release 420 mg capsules in pH 4.5 acetate buffer.

FIG. 3 compares the in-vitro dissolution of the diltiazem HCl extended release 420 mg capsules described in Example 12A vs. the commercially available diltiazem product Tiazac® extended release 420 mg capsules in pH 6.8 phosphate buffer.

FIG. 4 compares the in-vitro dissolution of the diltiazem HCl extended release 420 mg capsules described in Example 12A vs. the commercially available diltiazem product Tiazac® extended release 420 mg capsules in pH 1.2 simulated gastric fluid without pepsin for 2 hours followed by pH 6.8 phosphate buffer.

FIG. 5 shows the stability of the diltiazem HCl extended release 420 mg capsules described in Example 12A with regard to dissolution over three months under accelerated storage conditions.

FIG. 6 compares the in-vitro dissolution of the diltiazem HCl extended release 420 mg tablets described in Example 12B and 12C vs. Cardizem® LA extended release 420 mg tablets in pH 6.8 phosphate buffer.

### DETAILED DESCRIPTION OF THE INVENTION

### Definitions

As used herein, particular terms are defined as follows:

The singular forms “a,” “an,” and “the” include plural referents unless the context clearly dictates otherwise. Thus, for example, reference to “a drug” includes reference to one or more of drugs, and reference to “an excipient” includes reference to one or more excipients.

The term “active agent” refers to an agent that has measurable physiologic activity when administered to a subject in an effective amount. It is to be understood that the term “drug” is expressly encompassed by the present definition as many drugs and prodrugs are known to have specific physiologic activities. These terms of art are well-known in the pharmaceutical and medicinal arts.

The term “oral dosage form” refers to a formulation that is ready for administration to a subject via an oral route. Exemplary oral dosage forms include, but are not limited to, tablets, capsules, caplets, powders, pellets, beads, and granules. Oral dosage forms also include multi-layered tablets wherein each layer may contain a different drug. A dosage form can be a “unit dosage form,” which is intended to deliver one therapeutic dose per administration.

The term “core” refers to a plurality of granules, regardless of whether the granules are coated or uncoated, that are compressed together.

A “therapeutically effective amount” is an amount of a drug that is low enough to be non-toxic, yet sufficient to achieve a therapeutic result, including eliminating, reducing, and/or slowing the progression of a condition or symptom thereof. The therapeutically effective amount may depend on biological factors. Achieving a therapeutic result can be measured by a physician or other qualified medical personnel using objective evaluations known in the art, or it can be measured by individual, subjective patient assessment. The determination of a therapeutically effective amount is well within the ordinary skill in the art of pharmaceutical sciences and medicine. See, e.g., Meiner and Tonascia, “Clinical Trials: Design, Conduct, and Analysis,” Monographs in Epidemiology and Biostatistics, Vol. 8 (1986), incorporated herein by reference.

The term “substantially” refers to the complete or nearly complete extent or degree of a an item or result. For example, an object that is “substantially” enclosed would mean that the object is either completely enclosed or nearly completely enclosed. The exact allowable degree of deviation from absolute completeness may depend on the specific context. However, in general, the nearness of completion will be so as to have the same overall result as if total completion were obtained. In one aspect, “substantially” complete means being at least 75%, 90%, 95%, 98%, 99%, 99.5%, or 99.9% complete. The use of “substantially” is equally applicable when used to modifying a negative element, i.e., the substantial lack of an item or result. For example, a composition that is “substantially free of” particles would either completely lack particles, or so nearly completely lack particles that the effect would be the same as if it completely lacked particles. In one aspect, “substantially free” of a substance, e.g., a wetting agent, means having less than 10%, 5%, 2%, 1%, 0.5%, 0.3%, or 0.1% by weight of the substance. In another aspect, “substantially” unchanged, e.g., a substance that does not substantially change release, means the release is changed by less than 10%, 5%, 2%, 1%, 0.5%, 0.3%, or 0.1%.

The term “modified release” means that drug release is different from immediate release, i.e., dosage forms that releases about 60% or more of the drug in vivo within about 2 hours. Drug release may alternatively be measured in vitro by the dissolution of the drug in a dissolution medium according to methods known in the art. Examples of modified release profiles include, but are not limited to, sustained release, slow release, delayed release, and pulsatile release.

A “release-modifying coating” is a coating that alters the release rate of the drug from the dosage form, such that the release rate of a dosage form with a release-modifying coating is different from the release rate of an otherwise identical dosage form, but without the release-modifying coating, under identical conditions.

The term “subject” refers to a mammal to whom a pharmaceutical composition is administered. Exemplary subjects include humans, as well as veterinary and laboratory animals such as horses, pigs, cattle, dogs, cats, rabbits, rats, mice, and aquatic mammals.

Items may be presented herein in a common list for convenience. However, these lists should be construed as though each member of the list is individually identified as a separate and unique member. Thus, no individual member of such list should be construed as de facto equivalent to any other member of the same list solely based on their presentation in a common group without indications to the contrary.

Any numerical values recited herein include all values from the lower value to the upper value in increments of any measurable degree of precision. For example, if the value of a variable such as weight, weight percent, concentration, and the like is 1 to 90, specifically from 20 to 80, and more specifically from 30 to 70, it is intended that values such as 15 to 85, 22 to 68, 43 to 51, 30.3 to 32, etc., are expressly enumerated in this specification. In other words, all possible combinations of numerical values between the lowest value and the highest value enumerated are to be considered to be expressly stated in this application in a similar manner. This same principle applies to ranges reciting only one numerical value as a minimum or a maximum. Furthermore, such an interpretation should apply regardless of the breadth of the range or the characteristics being described.

### Modified Release Diltiazem

In one embodiment, a modified release diltiazem pharmaceutical composition is provided comprising:
a) a plurality of individual granules comprising a therapeutically effective amount of diltiazem hydrochloride and one or more pharmaceutically acceptable binders, wherein the individual granules are substantially free of a wetting agent; and
b) a release-modifying coating comprising either 1) one or more neutral acrylate polymers or 2) one or more water-insoluble cellulosic polymers, but not both; and wherein
   i) the individual granules are coated with the release-modifying coating to provide coated granules, or
   ii) the plurality of individual granules are compressed together to provide a core, and the core is coated with the release-modifying coating.

### Diltiazem Granules

The modified release diltiazem pharmaceutical compositions described herein include granules containing diltiazem, preferably as a pharmaceutically acceptable salt, particularly diltiazem hydrochloride.

The term “granule” encompasses any shape of particle, including irregularly shaped particles and/or spherical particles. The granules can be any suitable size, e.g., about 0.1 mm to about 1.0 mm. In one embodiment, granules size is about 100 μM to about 1200 μM, about 100 μM to about 1100 μM, about 150 μM to about 600 μM, or about 100 μM to about 400 μM as measured by methods well known in the art.

The plurality of granules (in aggregate) contains a therapeutically effective amount of diltiazem. More particularly, the plurality of granules forms a unit dosage form containing a therapeutically effective amount of diltiazem. For example, the plurality of granules can contain about 60 to about 500 mg, preferably about 120 mg to about 420 mg of diltiazem hydrochloride. In one embodiment, the plurality of granules contains about 60 mg, about 90 mg, about 120 mg, about 180 mg, about 240 mg, about 300 mg, about 360 mg, or about 420 mg diltiazem hydrochloride.

In addition to the diltiazem, the granules contain one or more pharmaceutically acceptable binders. Exemplary pharmaceutically acceptable binders include, but are not limited to, acrylate polymers (e.g., an acrylate polymer that does not substantially change the release of diltiazem such as Eudragit® L30D-55 or Eudragit® L100-55), cellulosic polymers, starch, povidone, sodium starch glycolate, and crospovidone. Exemplary cellulosic polymers include, but are not limited to, microcrystalline cellulose (MCC), ethylcellulose (EC), propylcellulose, isopropylcellulose, methylcellulose, carboxymethyl cellulose, hydroxypropyl cellulose (HPC), hydroxypropyl methylcellulose (HPMC), and hydroxyethyl cellulose. In one embodiment, the granules include microcrystalline cellulose and hydroxypropyl methylcellulose. In another embodiment, the granules include a cellulosic polymer and an acrylate polymer.

In one embodiment, the one or more pharmaceutically acceptable binders constitute about 1% to about 15% by weight of the granules.

The pharmaceutical composition may also contain one or more other pharmaceutically acceptable excipients known in the pharmaceutical arts, either within the granules or as an extragranular excipient. Exemplary excipients include, but are not limited to: microcrystalline cellulose, dibasic calcium phosphate dihydrate, starch, sodium starch glycolate, crospovidone, croscarmellose sodium, magnesium stearate, lactose, maleic acid, colloidal silicon dioxide, talc, and glyceryl behenate.

The additional excipients, taken together, can constitute about 20% to about 80% by weight of the unit dosage form, e.g., tablet or capsule. In one embodiment, the excipients constitute about 30% to about 80%, about 40% to about 80%, or about 50% to about 80% by weight of the diltiazem mixture.

The inventors have surprisingly discovered that the inclusion of a wetting agent is unnecessary, thus enabling a more economical manufacturing process while still achieving a desirable release profile. In one embodiment, the granules are readily hydratable, even without a wetting agent or dissolution agent. Accordingly, in one embodiment, the granules are substantially free of a wetting agent, more preferably the granules do not include a wetting agent. Wetting agents are saccharose, mannitol, sorbitol; lecithins; polyvinylpyrrolidones; C₁₂₋₂₀ fatty acid esters of saccharose; xylose esters or xylites; polyoxyethylenic glycerides; esters of fatty acids and polyoxyethylene; sorbitan fatty acid esters; and polyglycides-glycerides and polyglycides-alcohols esters.

The granules can be prepared by any method known in the art including, but not limited to, wet granulation, dry granulation, extrusion and/or spheronization, and “bead layering” (the application of the active agent and excipients onto an inert bead, e.g., a nonpareil bead). In another embodiment, methods of preparing a modified release diltiazem pharmaceutical composition are also provided.

### Methods of Preparing Diltiazem Granules

In one embodiment, methods of preparation are provided that include the steps of: a) combining a therapeutically effective amount of diltiazem hydrochloride and one or more pharmaceutically acceptable binders, wherein the diltiazem mixture is substantially free of a wetting agent; and b) granulating the diltiazem mixture to provide diltiazem granules. Granulation can be wet or dry granulation. For example, diltiazem, one or more pharmaceutically acceptable binders, and one or more additional pharmaceutically acceptable excipients can be mixed thoroughly to achieve a substantially homogenous mixture. Mixing can be accomplished, for example, by high shear granulators (mixers, blenders, etc).

The resultant mixture of diltiazem, binder(s), and excipient(s) (herein referred to as the “diltiazem mixture”) can be further processed by granulation with a water-insoluble polymer, spheronization, extrusion, bead layering, and/or compression.

In one embodiment, the diltiazem mixture is granulated with a water-insoluble polymer to form granules containing the diltiazem mixture and a water-insoluble polymer.

In one embodiment, the water-insoluble polymer can be used as a solution or dispersion at a concentration of about 1-20% in a non-aqueous solvent such as ethanol, isopropanol, or a mixture thereof. In another embodiment, the water-insoluble polymer concentration is about 1% to about 10%, about 5% to about 15%, about 5% to about 10%, about 3% to about 8%, about 4% to about 7%, or about 6%. One exemplary water-insoluble polymer is ethylcellulose.

Preparing the granules may further include a drying step. Drying can improve content uniformity and ease of handling. Thus, in one embodiment, the granules containing diltiazem, binder(s), excipient(s), and a water-insoluble polymer are dried using conventional drying techniques (e.g., tray drier or fluid bed drier (FBP)).

In another embodiment, the diltiazem mixture is wetted to facilitate extrusion and/or spheronization. The extrusion and/or spheronization steps can be carried out using processes and equipment well known in the art. Spheronization yields spheroids that may be optionally sieved to optimize desired particle size.

In yet another embodiment, the diltiazem mixture can be applied to nonpareil beads. The nonpareil beads can be any inert bead, e.g., starch or sugar spheres such as nonpareil sugar beads of size #25-30 or #30-35. The drug mixture can be applied using any known technique. For example, diltiazem and binder(s) can be dissolved in water to form a drug-loading mixture, and the drug-loading mixture can be applied to the nonpareil beads using a rotogranulator with tangential coating or a conventional coating pan with powder spraying/layering.

After any of these preparation techniques, the granules can be compressed to form a core. Alternatively, the granules can be used to fill a soft or hard capsule shell. The granules and/or cores can be coated with a release-modifying coating as described below to provide a unit dosage form (e.g., tablet or capsule).

### Release-Modifying Coating

To modify the release rate of diltiazem, a release-modifying coating is applied. The release-modifying coating can be applied to individual granules to provide coated granules. Additionally or alternatively, the plurality of granules can be compressed to provide a core, and the core is coated with a release-modifying coating.

In any case, the release-modifying coating can constitute about 1% to about 30%, about 1% to about 15%, about 5% to about 15%, about 5% to about 12%, about 5% to about 10%, about 8% to about 15%, about 8% to about 12%, or about 8% to about 10% by weight of the pharmaceutical composition prior to coating.

In one embodiment, the release-modifying coating substantially surrounds the uncoated granules and/or core. In another embodiment, the release-modifying coating completely surrounds the uncoated granules and/or core.

The coating ingredients should be selected so as to provide acceptable mechanical strength to withstand the coating process as well as any post-coating processes, including, e.g., tableting, packaging, transportation, etc. The coating should demonstrate little attrition or breakage in the fluid bed. If coated granules will be subjected to compression, the coating should remain substantially intact without significant cracking. The more flexible the coating, the more mechanical stability would be expected.

### Release-Modifying Polymers

In one embodiment, the release-modifying coating is a single polymer system containing either a) one or more neutral acrylate polymers or b) one or more water-insoluble cellulosic polymers, but not both (although more than one polymer within each class may be used).

Preferably, the release-modifying coating permits the modulated release of the active agent independent of pH. Appropriate polymer selection takes into account the physicochemical properties of the active agent as well as the desired retardation effect.

Exemplary water-insoluble cellulosic polymers include, but are not limited to: methylcellulose, ethylcellulose, propylcellulose, hydroxyethyl cellulose, hydroxypropyl cellulose, hydroxypropyl methylcellulose, and carboxymethylcellulose. In one embodiment, the water-insoluble cellulosic polymer is ethylcellulose.

Exemplary neutral acrylate polymers include, but are not limited to: polymers of methacrylate (e.g., Eudragit® NE40D and Eudragit® NE30D), methyl methacrylate, ethyl acrylate; copolymers of ethyl acrylate and methyl methacrylate; and methacrylate copolymers with trimethylammonio-ethylmethacrylate groups (e.g., Eudragit® RS30D-100 and Eudragit® RL30-100). In one embodiment, the release-modifying coating includes a neutral polymer of methacrylate. In one embodiment, the release-modifying coating includes a polymer of methacrylate, a methacrylate copolymer with trimethylammonio-ethylmethacrylates, or a mixture thereof. In another embodiment, the release-modifying coating includes a copolymer of ethyl acrylate and methyl methacrylate, e.g., having a ratio of ethyl acrylate to methyl methacrylate of about 2:1, (e.g., Eudragit® NE40D). Ethylacrylate methylmethacrylate copolymer with neutral ester groups does not have any functional ionic group; it swells in aqueous media independent of pH without dissolving.

Neutral polymers can advantageously be combined with both ionic and nonionic active agents.

To apply the coating, the release-modifying polymer is combined with an aqueous or non-aqueous solvent to produce a release-modifying mixture. Exemplary non-aqueous solvents include, but are not limited to: methanol, ethanol, isopropanol, acetone, or mixtures thereof. The release-modifying mixture can be, e.g., a solution, suspension, or emulsion. In one embodiment, the release-modifying mixture is ethyl cellulose in methyl alcohol having a viscosity of 100 cP.

The concentration of the release-modifying polymer in the release-modifying mixture can be about 0.5% to about 10%, about 1% to about 10%, about 2% to about 8%, about 2% to about 6%, about 1% to about 5%, about 1% to about 3%, or about 2% to about 3%. In one embodiment, the release-modifying mixture contains about 1% to about 2.5% ethylcellulose.

The release-modifying coating can also include one or more other excipients as is known in the art such as lubricants, flow promoting agents, plasticizers, anti-tacking agents, natural and synthetic flavorings, and natural and synthetic colorants. Specific exemplary coating excipients include, but are not limited to: polyethylene glycol, polyvinylpyrrolidone, talc, magnesium stearate, glyceryl behenate, stearic acid, and titanium dioxide.

In one embodiment, the release-modifying coating includes a plasticizer. Exemplary plasticizers include, but are not limited to, triethyl citrate and dibutyl sebacate.

In another embodiment, the release-modifying coating does not include a plasticizer. For example, neutral methacrylate polymers are advantageously flexible, soft, and have a low glass transition temperature. Accordingly, in some embodiments, neutral acrylate polymers can form a flexible coating even without a plasticizer.

In one embodiment, the release-modifying coating includes an anti-tacking agent. Exemplary anti-tacking agents include, but are not limited to: talc, glycerol monostearate (GMS), micronized silica, and magnesium stearate. The softer the polymer, the higher the amount of anti-tacking agent is recommended.

In one embodiment, the anti-tacking agent is talc. Talc can be included at a 100% (w/w) level calculated on dry polymer. Generally, talc needs to be suspended in water using a high shear mixer before being added to the release-modifying mixture.

In another embodiment, the anti-tacking agent is GMS, which advantageously affords purity and process advantages.

In yet another embodiment, the anti-tacking agent is micronized silica. Micronized silica can be used in quantities of about 10% to about 30% (w/w) of polymer. Micronized silica generally makes the coating more permeable, and, hence, can be applied to modify drug release.

In still another embodiment, the anti-tacking agent is magnesium stearate. Magnesium stearate is more effective than talc and often provides good sealing of the film coatings and low permeability. However, it is preferably used in organic polymer solutions, because coagulation or thickening may occur in aqueous solution.

### Coating Processes

Coatings, release-modifying or otherwise, can be applied using methods well known in the art. Granules, whether irregularly shaped particles or roughly spherical particles, can both be coated using similar methods known in the art. For example, coating can be accomplished in a conventional coating pan or a fluid bed coater with a bottom spray.

The coating process can be repeated as many times as necessary to achieve a coating thickness on the granules and/or cores such that the oral dosage form made therefrom achieves the desired in vitro and in vivo characteristics. In one embodiment, coating cycles are performed as many times as necessary to provide weight ratio of granules/cores to coating of about 20:1 to about 1:5, preferably about 5:1 to about 1:3. Alternatively, coating cycles are performed as many times as necessary to reach a coating thickness of about 10 μM to about 500 μM, about 200 μM to about 400 μM, or greater than 50 μM.

The release-modifying mixture can be applied alone, or together with a binding agent. The binding agent can be applied prior to, preferably immediately prior to, or together with, the release-modifying mixture. The binding agent can be provided as a solution, suspension, or powder. When the binding agent is provided as a powder, contact with the release-modifying mixture may at least partially dissolve the binder so as to form a solution or suspension. The binding agent then forms a binding film on the granules/cores to be coated, thus facilitating the adherence of the polymer. When the binding agent is provided as a solution or suspension, the binding agent wets the granules/cores, thereby facilitating the adherence of powdered polymeric particles.

The binding agent can mixed with a suitable solvent to form a binding mixture. Exemplary binding agents include, but are not limited to: vinyl polymers (e.g., polyvinylpyrrolidone, polyvinyl alcohol); cellulosic polymers (e.g., HPMC, HEC, HPC); acrylic polymers and copolymers (e.g., methacrylic acid polymers, ethyl acrylate-methylmethacrylate copolymers); natural or synthetic gums (e.g., guar gum, arabic gum, xanthan gum); proteins (e.g., gelatin), and carbohydrates (e.g., pectin). In one embodiment, the binding mixture contains polyvinylpyrrolidone. Suitable solvents are preferably capable of substantially completely solubilizing the specific binding agent(s) selected, and are pharmaceutically and biologically acceptable for ingestion. Suitable solvents will be readily determinable by those skilled in the art. In one embodiment, the solvent for the binding mixture is water.

The binding mixture can be used to wet the granules/cores using any technique known in the art. For example, granules can be wetted by rotating the granules in the binding mixture solution. Preferably, the granules are wetted using conventional automated pan coating equipment, wherein the granules are sprayed with the binding mixture while rotating in the pan.

The binding mixture should be of sufficient viscosity to enable wetting by such conventional techniques. Cellulosic polymers such as ethylcellulose (EC), hydroxypropyl methylcellulose (HPMC), and hydroxyethyl cellulose (HEC) can be included in the binding mixture to modify the viscosity. Each of these cellulosic polymers is available in variable forms (e.g., having varying viscosity, weight, and/or solubility). The binding mixture can contain one or more forms of a particular polymer and/or one or more different polymers. As used herein, when a polymer is named, it is meant to encompass one or more forms of that particular polymer.

In one embodiment, the binding mixture contains more than one form of HPMC. For example, the binding mixture can contain:
a) HPMC having i) substitution corresponding to about 30% methoxyl and about 10% hydroxypropoxyl groups by weight percent, and ii) a nominal viscosity at 20° C. of a 2% aqueous solution ranging from about 5 to about 100 mPa·s (e.g., METHOCEL® E5); and
b) HPMC having i) substitution corresponding to about 20% methoxyl and about 8% hydroxypropoxyl groups by weight percent, and ii) a nominal viscosity at 20° C. of a 2% aqueous solution ranging from about 4,000 to about 100,000 mPa·s (e.g., METHOCEL® K15M).

In one embodiment, the release-modifying coating constitutes about 1% to about 30%, about 1% to about 15%, about 5% to about 15%, about 1% to about 10%, about 1% to about 5%, about 1% to about 4%, about 1% to about 3%, or about 1% to about 2% by weight of the pharmaceutical composition. In another embodiment, the release-modifying coating is applied to a core, and the release-modifying coating constitutes about 1% to about 30%, preferably about 1% to about 15%, about 0.5% to about 5%, or about 0.5%, about 0.75%, about 1.0%, about 1.5%, about 2.0%, or about 3.0% by weight of the pharmaceutical composition, e.g., the coated tablet. In yet another embodiment, the release-modifying coating is applied to uncompressed granules, and the release-modifying coating constitutes about 5% to about 15% by weight of the pharmaceutical composition.

In one embodiment, the pharmaceutical composition is a tablet. Regardless of the process for preparing the tablet, the tablet can be coated with a cosmetic film, which does not substantially change the release of the drug from the dosage form. A cosmetic film can be made from, e.g., HPMC or wax. Similarly, granules coated with a release-modifying coating can also be coated with a cosmetic film.

In another embodiment, the pharmaceutical composition is a capsule. An exemplary capsule unit dosage form contains a hard or soft-gelatin capsule shell filled with coated granules.

The unit dosage forms (e.g., tablet, capsule) can be scored to facilitate dosing regimens.

### Additional Therapeutic Agents

The pharmaceutical compositions can also include one or more additional therapeutic agents. Exemplary additional therapeutic agents include, but are not limited to, cardiovascular agents and nonsteroidal anti-inflammatory agents (e.g., aspirin, diclofenac, ibuprofen, ketoprofen, piroxicam). Exemplary cardiovascular agents include, but are not limited to, ACE inhibitors, antihyperlipidemic agents, calcium channel blockers other than diltiazem, beta-blockers, antiplatelet agents (e.g., dipyridamole, aspirin, clopidogrel). Exemplary anti-hyperlipidemic agents include, but are not limited to, statins (e.g., fluvastatin, simvastatin, atorvastatin), bile acid sequestrants (e.g., colesevelam, cholestyramine, colestipol), fibrates and fibrate-related compounds (e.g., fenofibrate, gemfibrozil).

The additional therapeutic agent(s) can be formulated to provide any release profile including immediate release, rapid disintegration, modified release, delayed release, sustained release, slow release, pulsatile release, etc. The additional therapeutic agent(s) can be uniformly mixed with the diltiazem; in contact with the diltiazem as in a bilayer, multilayer, or coat/core format; or isolated from the diltiazem as in a bilayer, multilayer, or coat/core format including a barrier layer.

### Methods

In another embodiment, the invention provides methods for preparing a modified release diltiazem pharmaceutical composition as described above.

In yet another embodiment, modified release diltiazem pharmaceutical compositions prepared by the processes described above are provided.

In a preferred embodiment, the modified release diltiazem pharmaceutical composition is a unit dosage form that has a desirable in vitro dissolution profile and/or provides desirable in vivo blood plasma concentrations when administered to a mammal. In one embodiment, the unit dosage form provides peak blood plasma concentrations at about 6 hours after administration to a mammal. In another embodiment, the unit dosage form exhibits an AUC, Cₘₐₓ, and/or Tₘₐₓ that is about 80% to about 125% of the AUC, Cₘₐₓ, and/or Tₘₐₓ exhibited by Cardizem LA® or Tiazac®. In another embodiment, the unit dosage form is bioequivalent (as defined by the FDA) to Cardizem LA® or Tiazac®.

In a preferred embodiment, the unit dosage forms described herein are suitable for enteric administration, e.g., oral administration. Accordingly, in one embodiment, a method of treatment is provided comprising administering to a subject the pharmaceutical composition, e.g., a unit dosage form, described herein. The pharmaceutical composition can be used to treat cardiovascular disorders including, but not limited to: hypertension, angina, migraine, and hypertrophic subaortic stenosis. In one embodiment, a kit is provided including one or more unit dosage forms and appropriate labeling for interstate commerce.

The methods of treatment described herein can further include co-administering one or more additional therapeutic agents. The diltiazem and the additional therapeutic agents can be co-formulated as described above, or they can be co-administered (simultaneously or consecutively in any order) as separate pharmaceutical formulations.

### EXAMPLES

Without further elaboration, it is believed that one skilled in the art using the preceding description can utilize the invention to the fullest extent. The following examples are illustrative only, and not limiting of the disclosure in any way whatsoever. All percentages are in percent by weight of the formulation unless otherwise indicated.

### Example 1

Wet granulation: 609.69 g of diltiazem HCl and 281.02 g of microcrystalline cellulose (MCC) were loaded into a rapid mixing granulator (RMG) and mixed. A binder solution was prepared by dissolving 9.29 g of hydroxypropyl methylcellulose (HPMC E5 LV) in 261 ml of purified water. The RMG was turned on and run for about 5 min at 75 RPM. The RMG Chopper was then started and run at 207 RPM while adding the binder solution at a rate of 50 ml per min to yield diltiazem granules.

Extrusion and spheronization: The resulting diltiazem mixture was extruded through an extruder using a 0.8 mm screen to make round, long, threaded, plain extrudate. The extrudate was spheronized using a spheronizer having a 2.2 mm Pixture spheronization plate to provide round spheroids. The spheroids were discharged from the spheronizer and dried using a tray dryer at 60° C. for 5 hrs. The spheroids were dried until the moisture content was no more than (NMT) 1.0%. The spheroids were then passed through number 16 and number 25 screens.

Release-modifying coating: A release-modifying mixture was prepared by mixing 63 g of ethylcellulose N 100 in 1600 ml methyl alcohol using a magnetic stirrer for 120 min. 12.60 g of triethyl citrate was added, and mixing was continued for 10 min or more.

Bottom spray coating: The spheroids were coated with the release-modifying mixture in a fluid bed processor using bottom spray. The temperature was maintained at about 40° C.

Blending: 52.64 g of the coated spheroids were blended with 22.97 g of microcrystalline cellulose (MCC) and 22.97 g of polyethylene glycol (PEG) for 5 min, then lubricated with 0.71 g of magnesium stearate and 0.71 g purified talc for 3 min.

Tableting: The lubricated blend was compressed into tablets using a capsule shape 0.3750×0.7500 punch. Tablet weight was 1285 mg.

### Example 2

A modified release diltiazem tablet was prepared according to the processes of Example 1. For the diltiazem granules, 406.46 g of diltiazem HCl was mixed with 187.35 g of MCC, and 10.0 g of Eudragit® L30D-55 in 160 ml of purified water was used as the binder solution.

### Example 3

### Modified-Release Diltiazem by Granulation

Wet granulation: 406.46 g of diltiazem HCl, 157.35 g of MCC, and 36.0 g of methylcellulose were loaded into an RMG and mixed. A binder solution was prepared by mixing 2.1 g of methacrylic acid in 150 ml of purified water. The RMG was turned on and run for about 5 min at 100 RPM. The RMG Chopper was then started and run at 281 RPM while adding the binder solution at a rate of 30 ml per min. The diltiazem mixture was granulated for one more minute after the addition of the binder solution to yield diltiazem granules.

Drying: The granules were dried using a tray dryer at 60° C. for 8 hrs. The granules were dried until the moisture was NMT 1.0%. The granules were then passed through a number 18 screen.

Blending: 145.00 g of the granules were blended with 25.95 g of MCC, 75.50 g PEG, 2.03 g of magnesium stearate, and 2.03 g of talc to yield a lubricated blend.

Tableting: The lubricated blend was compressed to form cores using a capsule shape 0.3750×0.7500 punch. Tablet weight was 1100 mg.

Release-modifying coating: A release-modifying mixture was prepared by mixing 4 g of ethyl cellulose N 100 in 133 ml of isopropyl alcohol using a magnetic stirrer for 120 min. 0.50 g of triethyl citrate was added, and mixing was continued for 10 min or more.

Coating: The cores were coated with the release-modifying coating to reach a coating weight of about 0.5%, 0.75%, 1.0%, 1.5%, 2.0%, and 3.0% by weight of the coated tablet dosage form.

### Example 4

Wet granulation: 135.49 g of diltiazem HCl and 52.45 g of MCC were loaded into an RMG and mixed. A binder solution was prepared by mixing 0.9 g of methacrylic acid in 150 ml of purified water. The RMG was turned on and run for about 5 min at 113 RPM. The RMG Chopper was then started and run at 281 RPM while adding the binder solution at a rate of 30 ml per min. The diltiazem mixture was granulated for one more minute after the addition of the binder solution to yield diltiazem granules.

Drying: The granules were dried using a tray dryer at 70° C. for 8 hrs. The granules were dried until the moisture was NMT 1.0%. The granules were then passed through a number 18 screen.

Blending: The granules were blended with varying excipients to form lubricated blends as follows:
a) 48.50 g of the granules were blended with 40.00 g of methyl cellulose, 10.69 g of lactose granules, 0.81 g of magnesium stearate, and 0.81 g of talc.
b) 48.50 g of the granules were blended with 10.00 g of HPMC K4M, 30.00 g of HPMC K100M, 10.69 g of lactose granules, 0.81 g of magnesium stearate, and 0.81 g of talc.
c) 48.50 g of the granules were blended with 5.00 g of HPMC K4M, 10.00 g of HPMC K15M, 25.00 g of HPMC K100M, 10.69 g of lactose granules, 0.81 g of magnesium stearate, and 0.81 g of talc.
d) 16.98 g of the granules were blended with 3.50 g of methyl cellulose, 7.00 g of xanthan gum, 3.50 g of guar gum, 3.74 g of lactose granules, 0.29 g of magnesium stearate, and 0.29 g of talc.

Each of the lubricated blends was compressed to form cores as described in Example 3, except the tablet weight was 900 mg. The cores were coated using the release-modifying coating and process described in Example 3.

### Example 5

Wet granulation: Wet granulation was performed according to the process of Example 1. For the diltiazem granules, 677.4 g of diltiazem HCl was mixed with 302.0 g of MCC, and 20.6 g of HPMC E5 LV in 261 ml of purified water was used as the binder solution.

Extrusion and spheronization were performed using the process of Example 1, except using a 3.25 mm Pixture spheronization plate.

Release-modifying coating: 18.7 g of talc was added into 23.2 ml of water under stirring until it formed a uniform suspension. 2.00 g of simethicone was added. 37.5 g of a copolymer of ethyl acrylate and methyl methacrylate (2:1) were added with continued stirring. The coating was applied as described by Example 1.

### Example 6

A diltiazem formulation was prepared as described in Example 1, except using the release-modifying coating of Example 5.

### Example 7

A diltiazem formulation was prepared as described in Example 2, except using the release-modifying coating of Example 5.

### Example 8

A diltiazem formulation was prepared as described in Example 3, except using the release-modifying coating of Example 5.

### Example 9

A diltiazem formulation was prepared as described in Example 4, except using the release-modifying coating of Example 5.

### Example 10

A diltiazem formulation was prepared as described in Example 6, except using a 3.25 mm Pixture spheronization plate.

### Example 11

### Modified-Release Diltiazem Capsule

Diltiazem HCl (210 mg) and 10 mg of talc are mixed in an RMG. Diltiazem HCl (210 mg) and Hypromellose 5 cP (20 mg) are added in a sufficient amount of purified water to make a 25% solution based on the concentration Hypromellose. The diltiazem mixture is coated onto nonpareil sugar beads (170 mg) of size #25-30 or #30-35. The diltiazem mixture is coated onto the nonpareil beads in a rotogranulator with tangential coating or a conventional coating pan with powder spraying/layering. Alternatively, a Glatt GPCG 3.1 fluid bed coater can be used. The drug-coated beads are then film-coated with a solution of ethylcellulose 100 cP (24.80 mg) in methyl alcohol (1.24 mL, which is not present in finished dosage form) with dibutyl sebacate (2.48 mg) as plasticizer in a conventional coating pan. Size “00” capsules are filled with a sufficient amount of coated beads so that each capsule contains 420 mg of diltiazem HCl.

### Example 12

### Dissolution Profiles

### Example 12A

Modified-release diltiazem capsules were prepared using coated granules prepared as follows:

Diltiazem Granules

| | | Ind. Qty. | Total Qty. | Qty./Dose | Qty./Batch |
|---|---|---|---|---|---|
| Ingredient | Function | (% w/w) | (% w/w) | (mg) | (g) |
| Diltiazem Hydrochloride | Active | 67.74 | 65.93 | 420.00 | 2032.26 |
| Emcocel 90M (MCC) | Diluent | 31.23 | 30.39 | 193.60 | 936.75 |
| Methocel E5 LV Premium | Binder | 1.03 | 1.01 | 6.40 | 30.99 |
| (Polymer:Water ratio of | | | | | |
| 96.67:3.43) | | | | | |
| Purified Water | Solvent | | | | 870 ml |
| Total | | 100.00 | | 620.00 | 3000.00 |

The diltiazem granules were sifted through number 14 and number 20 screens to yield 1500.00 of sifted granules. Then the following release-modifying coating was applied to the granules, so as to yield a 2.5% weight increase.

Modified-Release Coating

| | | Ind. Qty. | Total Qty. | Qty./Dose | Qty./Batch |
|---|---|---|---|---|---|
| Ingredient | Function | (% w/w) | (% w/w) | (mg) | (g) |
| Aqualon N100 | Release-modifying | 2.50 | 2.43 | 15.50 | 37.50 |
| (Ethyl cellulose NF) | polymer | | | | |
| Dibutyl Sebacate NF | Plasticizer | 0.25 | 0.24 | 1.55 | 3.75 |
| Methyl Alcohol NF | Solvent | | | | 1483.13 |
| Total | | | 100.00 | 637.05 | 1541.25 |

The coated granules were used to fill capsule shells to provide oral dosage units of 420 mg diltiazem HCl.

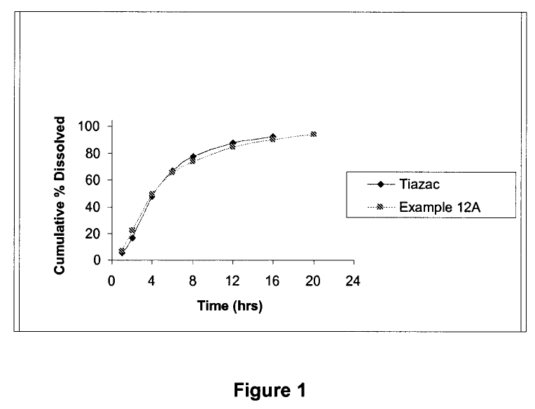

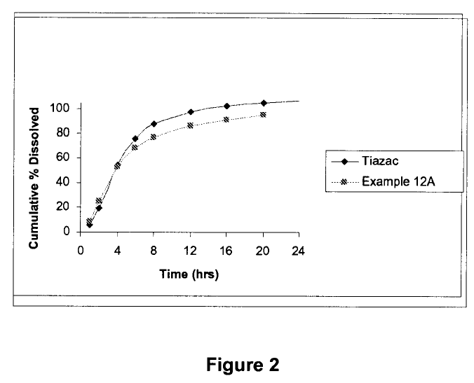

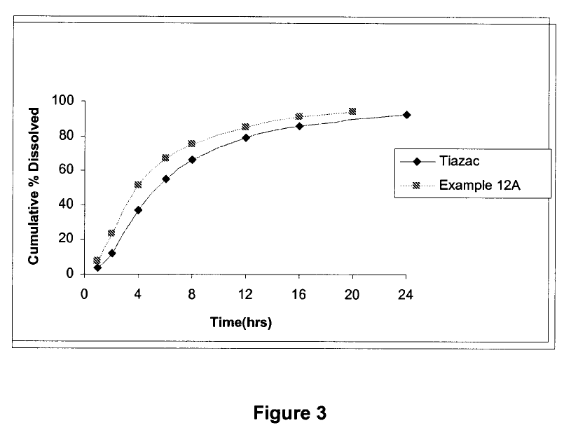

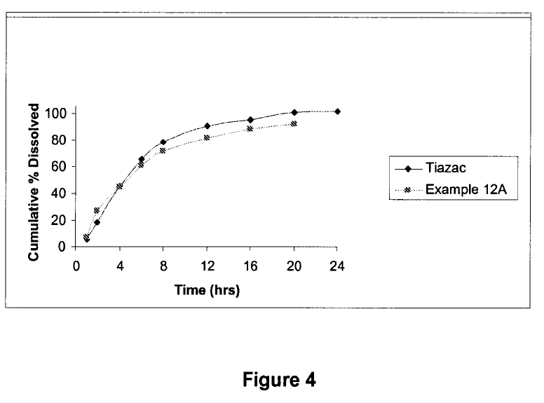

The modified-release diltiazem capsules were subjected to in-vitro dissolution analysis using the procedures of USP 29. More specifically, the capsules were tested using 900 mL of dissolution medium, using Apparatus 2 at RPM 100 at 37° C. The readings were measured using a UV detector operating at 237 nm. The samples were collected at 1, 2, 4, 6, 8, 12, 16, 20, and 24 hrs. The dissolution media included: simulated gastric fluid at pH 1.2, acetate buffer at pH 4.5, phosphate buffer at pH 6.8, and simulated gastric fluid at pH 1.2 followed by phosphate buffer at pH 6.8. The results are shown in Tables 1-4 and FIGS. 1-4, respectively.

TABLE 1
Comparative in-vitro dissolution of diltiazem HCl extended
release 420 mg capsules vs. the commercially available diltiazem
product Tiazac ® extended release 420 mg capsules
in pH 1.2 simulated gastric fluid without pepsin.

| Time (hrs) | Tiazac ® | Ex. 12A |
|---|---|---|
| 1 | 5.16 | 6.60 |
| 2 | 16.9 | 22.10 |
| 4 | 47.86 | 49.10 |
| 6 | 66.84 | 65.80 |
| 8 | 77.62 | 74.20 |
| 12 | 88.14 | 84.80 |
| 16 | 92.54 | 90.50 |
| 20 | | 94.00 |
| 24 | | |

TABLE 2
Comparative in-vitro dissolution of diltiazem HCl extended
release 420 mg capsules vs. the commercially available
diltiazem product Tiazac ® extended release
420 mg capsules in pH 4.5 acetate buffer.

| Time (hrs) | Tiazac ® | Ex. 12A |
|---|---|---|
| 1 | 5.9 | 8.6 |
| 2 | 19.3 | 25.3 |
| 4 | 54.5 | 52.8 |
| 6 | 75.9 | 68.6 |
| 8 | 87.2 | 76.7 |
| 12 | 97.9 | 85.9 |
| 16 | 102.8 | 90.8 |
| 20 | 105 | 94.7 |
| 24 | 106.5 | |

TABLE 3
Comparative in-vitro dissolution of diltiazem HCl extended
release 420 mg capsules vs. the commercially available
diltiazem product Tiazac ® extended release
420 mg capsules in pH 6.8 phosphate buffer.

| Time (hrs) | Tiazac ® | Ex. 12A |
|---|---|---|
| 1 | 3.57 | 7.3 |
| 2 | 12.34 | 23.4 |
| 4 | 37.1 | 51.3 |
| 6 | 55.07 | 67 |
| 8 | 66.54 | 75.5 |
| 12 | 79.14 | 85.3 |
| 16 | 85.77 | 90.7 |
| 20 | | 93.9 |
| 24 | 92.59 | |

TABLE 4
Comparative in-vitro dissolution of diltiazem HCl extended
release 420 mg capsules vs. the commercially available diltiazem
product Tiazac ® extended release 420 mg capsules
in pH 1.2 simulated gastric fluid without pepsin for 2 hours
followed by pH 6.8 phosphate buffer.

| Time (hrs) | Tiazac ® | Ex. 12A |
|---|---|---|
| 1 | 5.7 | 7.1 |
| 2 | 18.5 | 27.1 |
| 4 | 45.5 | 44.4 |
| 6 | 65.2 | 60.6 |
| 8 | 78.2 | 71.2 |
| 12 | 90.3 | 81.5 |
| 16 | 95.3 | 88 |
| 20 | 100.2 | 92.2 |
| 24 | 101.2 | |

### Examples 12B and C

A modified release diltiazem tablet was prepared according to the general process described in Example 3. For the diltiazem granules, 270.98 g of diltiazem HCl, 103.84 g of MCC, and 24.00 g of methylcellulose were mixed, and 24.00 g of an anionic copolymer of methacrylic acid and methacrylate in 261 ml of purified water was used as the binder solution. For the blending step, 97.30 g of the granules were blended with 45.06 g of MCC, 54.40 g PEG, 1.62 g of magnesium stearate, and 1.62 g of talc to yield a lubricated blend. The tablet weight was 1275 mg. The release-modifying coating was prepared from 2.0225 g of ethyl cellulose N 4, 0.225 g of methylcellulose in 85 ml of isopropyl alcohol, and 0.15 g of triethyl citrate. The cores were coated with the release-modifying coating to reach a coating weight of about 5.0% (Example 12B), 7.5%, 10.0% (Example 12C), and 12.5% by weight of the coated tablet dosage form.

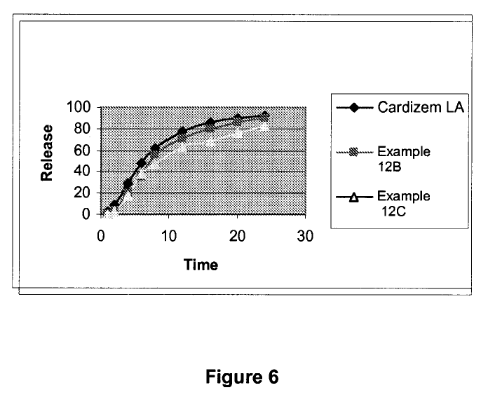

FIG. 6 shows a comparison of the in vitro dissolution profile of diltiazem HCl extended release 420 mg tablets according to Examples 12B and 12C vs. the commercially available diltiazem product Cardizem® LA extended release 420 mg tablets in pH 6.8 phosphate buffer.

The dissolution profiles demonstrate that the inventive diltiazem HCl formulations have release rates that are similar to the commercially available diltiazem products Tiazac® and Cardizem® LA.

### Example 13

### Stability

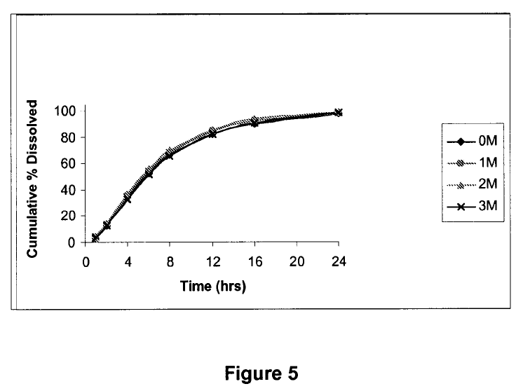

The modified-release diltiazem capsules of Example 12A were also subjected to stability tests under accelerated storage conditions over three months. The modified-release diltiazem capsules demonstrated acceptable stability at 40° C., 75% relative humidity. The capsules were tested using 900 mL of USP water as the dissolution medium, using Apparatus 2 at RPM 100 at 37° C. The results are shown in Table 5 and FIG. 5.

TABLE 5
Dissolution of inventive diltiazem HCl
extended release 420 mg capsules

| Time (hrs) | 0M | 1M | 2M | 3M |
|---|---|---|---|---|
| 1 | 2.9 | 3.70 | 3.74 | 3.00 |
| 2 | 12.51 | 13.71 | 13.97 | 12.54 |
| 4 | 34.2 | 35.51 | 36.64 | 33.24 |
| 6 | 52.83 | 54.17 | 55.65 | 51.52 |
| 8 | 66.42 | 68.22 | 69.62 | 65.73 |
| 12 | 82.27 | 84.55 | 85.76 | 82.33 |
| 16 | 90.25 | 92.65 | 93.67 | 91.00 |
| 24 | 98.17 | 98.16 | 98.81 | 98.91 |

### Example 14

### Bioequivalence

The bioavailability of the modified-release diltiazem capsules of Example 12A were compared to the bioavailability of the commercially available diltiazem product Tiazac® extended release 420 mg capsules as shown in Tables 6-7.

TABLE 6
Bioavailability under fasting conditions in a
two way crossover biostudy with n = 24.

| | Test | Ref | Test/Ref |
|---|---|---|---|
| | Mean | Mean | Ratio |
| Cₘₐₓ | 170.81 | 286.25 | 59.67 |
| AUC_{0-inf} | 3025.03 | 4000.83 | 75.61 |
| Tₘₐₓ | 7.08 | 7.19 | 98.49 |
| | Test G. | Ref G. | G Mean |
| | Mean | Mean | Ratio |
| Cₘₐₓ | 161.08 | 264.85 | 60.82 |
| AUC_{0-inf} | 2806.98 | 3729.60 | 75.26 |

TABLE 7
Bioavailability under non-fasting conditions in
a two way crossover biostudy with n = 22.

| | Test | Ref | Test/Ref |
|---|---|---|---|
| | Mean | Mean | Ratio |
| Cₘₐₓ | 235.23 | 323.22 | 72.78 |
| AUC_{0-inf} | 4023.42 | 4527.26 | 88.87 |
| Tₘₐₓ | 8.64 | 6.99 | 123.50 |
| | Test G. | Ref G. | G Mean |
| | Mean | Mean | Ratio |
| Cₘₐₓ | 224.82 | 311.69 | 72.13 |
| AUC_{0-inf} | 3866.71 | 4341.00 | 89.07 |

The examples given above are merely illustrative and are not meant to be an exhaustive list of all possible embodiments, applications, or modifications of the invention. Various modifications of the described methods and compositions will be apparent to those skilled in the art without departing from the scope and spirit of the invention.

The disclosures of all references and publications cited above are expressly incorporated by reference in their entireties to the same extent as if each were incorporated by reference individually.

What is claimed is:

1. A modified release diltiazem pharmaceutical composition comprising:
a) a plurality of individual granules comprising a therapeutically effective amount of diltiazem hydrochloride and one or more pharmaceutically acceptable binders, wherein the individual granules are substantially free of a wetting agent; and
b) a release-modifying coating comprising either 1) one or more neutral acrylate polymers or 2) one or more water-insoluble cellulosic polymers, but not both; and wherein
i) the individual granules are coated with the release-modifying coating to provide coated granules, or
ii) the plurality of individual granules are compressed together to provide a core, and the core is coated with the release-modifying coating.

2. The composition of claim 1, wherein the therapeutically effective amount of diltiazem hydrochloride is about 60 to about 500 mg.

3. The composition of claim 1, wherein the one or more pharmaceutically acceptable binders constitute about 1% to about 15% by weight of the granules.

4. The composition of claim 1, wherein one or more pharmaceutically acceptable binders is selected from the group consisting of: acrylate polymers, cellulosic polymers, starch, povidone, sodium starch glycolate, and crospovidone.

5. The composition of claim 4, wherein one or more pharmaceutically acceptable binders is a cellulosic polymer selected from the group consisting of: microcrystalline cellulose, ethylcellulose, propylcellulose, isopropylcellulose, methylcellulose, carboxymethylcellulose, hydroxypropyl cellulose, hydroxypropyl methylcellulose, and hydroxyethyl cellulose.

6. The composition of claim 1, wherein the release-modifying coating constitutes about 1% to about 30% by weight of the pharmaceutical composition.

7. The composition of claim 6, wherein the release-modifying coating constitutes about 5% to about 15% by weight of the pharmaceutical composition.

8. The composition of claim 1, wherein the release-modifying coating comprises one or more neutral acrylate polymers selected from the group consisting of: polymers of ethyl acrylate, polymers of methyl methacrylates, polymers of methacrylates, copolymers of ethyl acrylate and methyl methacrylate, and methacrylate copolymers with trimethylammonio-ethylmethacrylate.

9. The composition of claim 8, wherein the release-modifying coating comprises a copolymer of ethyl acrylate and methyl methacrylate in a ratio of 2:1.

10. The composition of claim 1, wherein the release-modifying coating comprises one or more water-insoluble cellulosic polymers selected from the group consisting of: ethylcellulose, methylcellulose, propylcellulose, hydroxyethyl cellulose, and hydroxypropyl methylcellulose.

11. The composition of claim 10, wherein the release-modifying coating comprises ethylcellulose.

12. The composition of claim 1, wherein the coated granules are compressed to form a core.

13. The composition of claim 12, wherein the core is coated with the release-modifying coating.

14. The composition of claim 13, wherein the core is further coated with a cosmetic coating.

15. The composition of claim 1, wherein the core is further coated with a cosmetic coating.

16. The composition of claim 1, wherein the coated granules are filled into a soft or hard capsule shell.

17. The composition of claim 1, wherein the granules are spheroids.

18. A process for preparing a modified release diltiazem pharmaceutical composition comprising the steps of:
a) combining a therapeutically effective amount of diltiazem hydrochloride and one or more pharmaceutically acceptable binders, wherein the diltiazem mixture is substantially free of a wetting agent;
b) granulating the diltiazem mixture to provide diltiazem granules;
c) preparing a release-modifying coating comprising either 1) one or more neutral acrylate polymers or 2) one or more water-insoluble cellulosic polymers, but not both; and
i) coating the plurality of individual granules with the release-modifying coating to form coated granules; or
ii) compressing the plurality of individual granules together to provide a core, and then coating the core with the release-modifying coating.

19. The process of claim 18, wherein granulating comprises wet granulation.

20. The process of claim 18, further comprising a step of extrusion.

21. The process of claim 18, wherein further comprising a step of spheronization.

22. The process of claim 18, further comprising compressing the coated granules.

23. The process of claim 18, further comprising filling a soft or hard capsule shell with the coated granules.

24. The process of claim 18, further comprising applying a cosmetic coating.

25. The process of claim 18, wherein one or more pharmaceutically acceptable binders is selected from the group consisting of: acrylate polymers, cellulosic polymers, starch, povidone, sodium starch glycolate, and crospovidone.

26. The process of claim 25, wherein one or more pharmaceutically acceptable binders is a cellulosic polymer selected from the group consisting of: microcrystalline cellulose, ethylcellulose, propylcellulose, isopropylcellulose, methylcellulose, carboxymethylcellulose, hydroxypropyl cellulose, hydroxypropyl methylcellulose, and hydroxyethyl cellulose.

27. The process of claim 18, wherein the release-modifying coating comprises one or more neutral acrylate polymers selected from the group consisting of: polymers of ethyl acrylate, polymers of methyl methacrylates, polymers of methacrylates, copolymers of ethyl acrylate and methyl methacrylate, and methacrylate copolymers with trimethylammonio-ethylmethacrylate.

28. The process of claim 27, wherein the release-modifying coating comprises a copolymer of ethyl acrylate and methyl methacrylate copolymer in a ratio of 2:1.

29. A modified release diltiazem pharmaceutical composition prepared by the process of claim 18.
